SEQUENCE LISTING (2 sequences)

SEQ ID NO: 1 - protein, 887 aa, Homo sapiens
MGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 426 aa, Artificial
  soluble AXL polypeptide-Fc fusion
EESPFVSNPGNITGARGLTGTLRCQLQVQGEPPEVHWLRDGQILELVDSTQTQVPLGEDEQGDWIVASQLRITSLQLSDTGQYQCLVFLGHQTFVSQPGYVRLEGLPYFLEEPEDRTVAANTPFNLSCQAQGPPEPVDLLWLQDAVPLATAPGHGPQRSLHVPGLNKTSSFSCEAHNAKGVTTSRTATITVLPQQGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2023/0084893 A1
(43) Pub. Date: Mar. 16, 2023

(54) METHODS OF TREATING CLEAR CELL RENAL CELL CARCINOMA (ccRCC) USING AXL DECOY RECEPTORS

(71) Applicant: Aravive Inc, Houston, TX (US)

(72) Inventors: Gail MCINTYRE, Lansdale, PA (US); Laura BONIFACIO, Durham, NC (US)

(21) Appl. No.: 17/790,282

(22) PCT Filed: Jan. 5, 2021
(86) PCT No.: PCT/US21/12176

§ 371 Date: Jun. 30, 2022

Related U.S. Application Data
(60) Provisional application No. 62/957,622, filed on Jan. 6, 2020.

(51) Int. Cl. A61K 38/17; C07K 14/82; A61K 31/167; A61P 35/00

(52) U.S. Cl. CPC A61K 38/177; C07K 14/82; A61K 31/167; A61P 35/00; C07K 2319/30

(57) ABSTRACT

Compositions and methods are provided for treating advanced clear cell renal cell carcinoma (RCC) in a mammal by administering a therapeutic dose of a pharmaceutical composition that inhibits AXL protein activity, for example by inhibition of the binding interaction between AXL and its ligand GAS6, in combination with a therapeutic dose of cabozantinib.

### RELATED APPLICATIONS

This application claims benefit of U.S. Provisional Application No. 62/957,622, filed on Jan. 6, 2020, incorporated in its entirety by reference herein.

### SEQUENCE LISTING

The instant application contains a Sequence Listing in the form of a “paper copy” (PDF File) and a file containing the referenced sequences (SEQ D NOS: 1 and 2) in computer readable form (ST25 format text file) which is submitted herein. The Sequence Listing is shown using standard three letter code for amino acids, as defined in 37 C.F.R. 1.822.

### STATEMENT REGARDING FEDERALLY SPONSORED RESEARCH OR DEVELOPMENT

This work was supported by Cancer Prevention & Research Institute of Texas, New Company Product Development Award DP150127. The State of Texas, USA, may have rights in any patent issuing on this application.

### TECHNICAL FIELD

Clear cell renal cell carcinoma (ccRCC) is the most common form of kidney cancer, accounting for more than 90% of all cases, and has been found to be nearly twice as common in men as in women. An estimated 65,000 new cases of ccRCC are diagnosed in the United States (US) each year, with a median age of 65 at diagnosis. Based on 1992-2005 SEER statistics, the incidence of ccRCC rose by 1.8% and 2.1% in white men and women, respectively, with the increase determined to be the result of a decrease in the average size of tumors at presentation. Early detection of small tumors, however, has not been associated with a decline in ccRCC-related mortality. Despite the introduction of new therapies in the last decade, 13,500 deaths occur annually in the US. These therapies have improved the median overall survival of patients, which currently exceeds two years, primarily due to advances the development of targeted systemic therapies. The five-year survival rate for patients with stage Ill and IV disease is ˜69% and 12% (American Cancer Society), respectively, with metastases at distant sites, including the lungs, lymph nodes, liver, bone and brain, being the principal cause of mortality.

The treatment options for advanced and metastatic ccRCC have expanded and include multiple anti-angiogenic drugs that target VEGF and its receptors, mTOR inhibitors, as well as immune checkpoint inhibitors. More than ten targeted and immunotherapies have received approval from the US Food and Drug Administration since 2005. Many more are currently being evaluated in clinical trials, providing multiple first- and second-line treatment options for patients with ccRCC. While clinical benefit is demonstrated, the complete response rate for these therapies is very poor (less than 10%), leaving most patients unresponsive and without other treatment options as therapeutic resistance develops.

As a result of recent approvals and advances in the therapeutic space, most ccRCC patients receive front-line combination treatment with either two immunotherapeutic (10) agents or a combination of anti-VEGF and 10 agent. Of the options, two front-line combinations containing Inlyta® (axitinib-a small molecule anti-VEGF) with Keytruda® (pembrolizumab) or another front-line 10/10 combination are used most commonly. In 2016, Cabometyx™ (cabozantinib (a small molecule targeting VEGF with a different tyrosine kinase profile) was approved by the Food and Drug Administration (FDA) in the United States for the treatment of advanced renal cell carcinoma (RCC) who have received prior antiangiogenic therapy. As such, cabozantinib is favored as a second line agent of choice as standard of care.

In spite of recent advances, there still exists an unmet need for the development of novel systemic therapies that achieve improvement in the three efficacy endpoints of progression-free survival (PFS), objective response rate, and overall survival in the treatment of renal cell carcinoma.

Patent documents Ser. Nos. 13/554,954; 13/595,936; 13/714,875; 13/950,111; 14/712,731; 14/650,852; 14/650,854; 14/910,565; US2011/022125; US2013/056435; US2012/069841; US2013/074809; US2013/074786; US2013/074796; US2015/0315553 are herein specifically incorporated by reference for all teachings.

### DISCLOSURE OF THE INVENTION

In one aspect, the present invention provides methods for the treatment of advanced clear cell renal cell carcinoma (ccRCC) in a human patient, comprising the administration of a soluble AXL polypeptide, according to a regimen determined to achieve stable disease/response and longer progression free survival (PFS) as compared to control.

In another aspect, the present invention provides methods for the treatment of advanced clear cell renal cell carcinoma (ccRCC) in a human patient, comprising the administration of a soluble AXL polypeptide in combination with cabozantinib according to a regimen determined to achieve stable disease/response and longer progression free survival as compared to control. In some embodiments, the soluble AXL polypeptide may offer additive or synergistic benefit to the therapeutic activity of cabozantinib.

In another aspect, the present invention provides methods for the treatment of advanced clear cell renal cell carcinoma (ccRCC) in a human patient who has received prior anti-angiogenic therapy, comprising the administration of a soluble AXL polypeptide in combination with cabozantinib, wherein progression-free survival (PFS) and one or both of overall survival (OS) and objective response rate (ORR) are extended as compared to patients who have received prior anti-angiogenic therapy. In some embodiments, the prior anti-angiogenic therapy is selected from the group consisting of axitinib, pazopanib, sorafenib, sunitinib, everolimus, temsirolimus, bevacizumab, interleukins, interferon-α, peginterferon, nivolumab, and atezolizumab. In some embodiments, the soluble AXL polypeptide may offer additive or synergistic benefit to the therapeutic activity of cabozantinib.

In some embodiments, the soluble AXL polypeptide is a soluble AXL variant polypeptide, wherein said soluble AXL variant polypeptide lacks the AXL transmembrane domain, lacks a functional fibronectin (FN) domain, has one or more Ig1 domain, has one or more Ig2 domain, and wherein said AXL variant polypeptide exhibits increased affinity of the AXL variant polypeptide binding to GAS6 compared to wild-type AXL.

In some embodiments, the soluble AXL polypeptide is a soluble AXL variant polypeptide, wherein said soluble AXL variant polypeptide lacks the AXL transmembrane domain, lacks a functional fibronectin (FN) domain, has one Ig1 domain, lacks a functional Ig2 domain and wherein said AXL variant polypeptide exhibits increased affinity of the AXL variant polypeptide binding to GAS6 compared to wild-type AXL.

In some embodiments, the AXL variant polypeptide is a fusion protein comprising an Fc domain. In some embodiments, the variant polypeptide lacks the AXL intracellular domain. In some embodiments, the soluble AXL variant polypeptide further lacks a functional fibronectin (FN) domain and wherein said variant polypeptide exhibits increased affinity of the polypeptide binding to GAS6. In some embodiments, the soluble AXL variant polypeptide comprises at least one amino acid modification relative to the wild-type AXL sequence.

In some embodiments, the soluble AXL variant polypeptide comprises at least one amino acid modification within a region selected from the group consisting of 1) between 15-50, 2) between 60-120, and 3) between 125-135 of the wild-type AXL sequence (SEQ ID NO:1).

In some embodiments, the soluble AXL variant polypeptide comprises at least one amino acid modification at position 19, 23, 26, 27, 32, 33, 38, 44, 61, 65, 72, 74, 78, 79, 86, 87, 88, 90, 92, 97, 98, 105, 109, 112, 113, 116, 118, or 127 of the wild-type AXL sequence (SEQ ID NO: 1) or a combination thereof.

In some embodiments, the soluble AXL variant polypeptide comprises at least one amino acid modification selected from the group consisting of 1) A19T, 2) T23M, 3) E26G, 4) E27G or E27K 5) G32S, 6) N33S, 7) T38I, 8) T44A, 9) H61Y, 10) D65N, 11) A72V, 12) S74N, 13) Q78E, 14) V79M, 15) Q86R, 16) D87G, 17) D88N, 18) 190M or 190V, 19) V92A, V92G or V92D, 20) 197R, 21) T98A or T98P, 22) T105M, 23) Q109R, 24) V112A, 25) F113L, 26) H116R, 27) T118A, 28) G127R or G127E, and 29) G129E and a combination thereof.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; and (d) glycine 127.

In some embodiments, the AXL variant polypeptide comprises amino acid changes relative to the wild-type AXL sequence (SEQ ID NO: 1) at the following positions: (a) aspartic acid 87 and (b) valine 92.

In some embodiments, the AXL variant polypeptide comprises amino acid changes relative to the wild-type AXL sequence (SEQ ID NO: 1) at the following positions: (a) glycine 32; (b) aspartic acid 87; (c) valine 92; (d) glycine 127 and (e) alanine 72.

In some embodiments, the AXL variant polypeptide comprises amino acid changes relative to the wild-type AXL sequence (SEQ ID NO: 1) at the following position: alanine 72.

In some embodiments, the AXL variant polypeptide glycine 32 residue is replaced with a serine residue, aspartic acid 87 residue is replaced with a glycine residue, valine 92 residue is replaced with an alanine residue, or glycine 127 residue is replaced with an arginine residue or a combination thereof.

In some embodiments, the AXL variant polypeptide residue aspartic acid 87 residue is replaced with a glycine residue or valine 92 residue is replaced with an alanine residue or a combination thereof.

In some embodiments, the AXL variant polypeptide alanine 72 residue is replaced with a valine residue.

In some embodiments, the AXL variant polypeptide glycine 32 residue is replaced with a serine residue, aspartic acid 87 residue is replaced with a glycine residue, valine 92 residue is replaced with an alanine residue, glycine 127 residue is replaced with an arginine residue or an alanine 72 residue is replaced with a valine residue or a combination thereof.

In some embodiments, the AXL variant comprises amino acid changes relative to the wild-type AXL sequence (SEQ ID NO: 1) at the following positions: (a) glutamic acid 26; (b) valine 79; (c) valine 92; and (d) glycine 127.

In some embodiments, the AXL variant polypeptide glutamic acid 26 residue is replaced with a glycine residue, valine 79 residue is replaced with a methionine residue, valine 92 residue is replaced with an alanine residue, or glycine 127 residue is replaced with an arginine residue or a combination thereof.

In some embodiments, the AXL variant polypeptide comprises at least an amino acid region selected from the group consisting of amino acid region 19-437, 130-437, 19-132, 21-121, 26-132, 26-121 and 1-437 of the wild-type AXL polypeptide (SEQ ID NO: 1), and wherein one or more amino acid modifications occur in said amino acid region.

In some embodiments, the AXL variant polypeptide comprises amino acid changes relative to the wild-type AXL sequence (SEQ ID NO: 1) at the following positions: (a) glycine 32; (b) aspartic acid 87; (c) alanine 72; and (d) valine 92.

In some embodiments, the AXL variant polypeptide glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein further comprising an Fc domain and wherein said AXL variant comprises amino acid changes relative to wild-type AXL sequence (SEQ ID NO:1) at the following positions: (a) glycine 32; (b) aspartic acid 87; (c) alanine 72; and (d) valine 92.

In some embodiments, the soluble AXL polypeptide is a fusion protein comprising an Fc domain and wherein glycine 32 is replaced with a serine residue, aspartic acid 87 is replaced with a glycine residue, alanine 72 is replaced with a valine residue, and valine 92 is replaced with an alanine residue, or a combination thereof.

In some embodiments, the soluble AXL polypeptide is a fusion protein comprising an Fc domain and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL polypeptide is a fusion protein comprising an Fc domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL polypeptide is a fusion protein comprising an Fc domain, lacks a functional FN domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant is a fusion protein comprising an Fc domain, lacks a functional FN domain, and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72 and (d) valine 92.

In some embodiments, the soluble AXL variant is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, and valine 92 is replaced with an alanine residue or a combination thereof.

In some embodiments, the soluble AXL polypeptide is a fusion protein comprising an Fc domain, lacks a functional FN domain, lacks an Ig2 domain, and wherein said AXL variant comprises amino acid changes relative to wild-type AXL sequence (SEQ ID NO:1) at the following positions: (a) glycine 32; (b) aspartic acid 87; (c) alanine 72; (d) valine 92; and (e) glycine 127.

In some embodiments, the soluble AXL variant is a fusion protein comprising an Fc domain, lacks a functional FN domain, lacks an Ig2 domain and wherein glycine 32 is replaced with a serine residue, aspartic acid 87 is replaced with a glycine residue, alanine 72 is replaced with a valine residue, valine 92 is replaced with an alanine residue, and glycine 127 is replaced with an arginine residue or a combination thereof.

In some embodiments, the soluble AXL variant polypeptide has an affinity of at least about 1×10⁻⁸ M, 1×10⁻⁹ M, 1×10⁻¹⁰ M, 1×10⁻¹¹ M or 1×10⁻¹² M for GAS6.

In some embodiments, the soluble AXL variant polypeptide exhibits an affinity to GAS6 that is at least about 5-fold stronger, at least about 10-fold stronger or at least about 20-fold stronger than the affinity of the wild-type AXL polypeptide.

In some embodiments, the soluble AXL variant polypeptide further comprises a linker. In some embodiments, the linker comprises one or more (GLY)₄SER units. In some embodiments, the linker comprises 1, 2, 3 or 5 (GLY)₄SER units. In some embodiments, the linker comprises 1 (GLY)₄SER unit.

In some embodiments, the soluble AXL polypeptide is a fusion protein comprising an Fc domain, a linker, lacks a functional FN domain, and having the amino acid sequence set forth in SEQ ID NO: 2 (hereinafter “AVB-S6-500).

In some embodiments, the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0, of about 19.5, of about 20.0, of about 20.5, of about 21.0, of about 21.5, of about 22.0, of about 22.5, of about 23.0, of about 23.5, of about 24.0, of about 24.5, of about 25.0, of about 25.5, of about 26.0, of about 26.5, of about 27.0, of about 27.5, of about 28.0, of about 28.5, of about 29.0, of about 29.5, and of about 30.0 mg/kg. In some embodiments, the soluble AXL variant polypeptide will be given as IV infusion over 30 or 60 minutes at a weekly dose of 10 mg/kg. In some embodiments, the soluble AXL variant polypeptide will be given as IV infusion over 30 or 60 minutes at a weekly dose of 5 mg/kg. In some embodiments, the soluble AXL variant polypeptide will be given as IV infusion over 30 or 60 minutes at a weekly dose of 2.5 mg/kg. In some embodiments, the soluble AXL variant polypeptide will be given as IV infusion over 30 or 60 minutes at a weekly dose of 1 mg/kg. In some embodiments, the soluble AXL variant polypeptide will be given as IV infusion over 30 or 60 minutes at a dose of 25 mg/kg every 14 days. In some embodiments, the soluble AXL variant polypeptide will be given as IV infusion over 30 or 60 minutes at a dose of 20 mg/kg every 14 days. In some embodiments, the soluble AXL variant polypeptide will be given as IV infusion over 30 or 60 minutes at a dose of 15 mg/kg every 14 days. In some embodiments, the soluble AXL variant polypeptide will be given as IV infusion over 30 or 60 minutes at a dose of 10 mg/kg every 14 days. In some embodiments, the soluble AXL variant polypeptide will be given as IV infusion over 30 or 60 minutes at a dose of 5 mg/kg every 14 days. In some embodiments, the soluble AXL variant polypeptide will be given as IV infusion over 30 or 60 minutes at a dose of 2.5 mg/kg every 14 days. In some embodiments, the soluble AXL variant polypeptide will be given as IV infusion over 30 or 60 minutes at a dose of 1 mg/kg every 14 days.

In some embodiments, the dose of cabozantinib to be co-administered to the patient along with the soluble AXL variant polypeptide is selected from the group consisting of 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, and 5 mg, once daily with fasting.

### MODE(S) FOR CARRYING OUT THE INVENTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

The terms “polypeptide,” “peptide” and “protein” are used interchangeably herein to refer to a polymer of two or more amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms “antibody” and “antibodies” are used interchangeably herein and refer to a polypeptide capable of interacting with and/or binding to another molecule, often referred to as an antigen. Antibodies can include, for example “antigen-binding polypeptides” or “target-molecule binding polypeptides.” Antigens of the present invention can include for example any polypeptides described in the present invention.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. All single letters used in the present invention to represent amino acids are used according to recognized amino acid symbols routinely used in the field, e.g., A means Alanine, C means Cysteine, etc. An amino acid is represented by a single letter before and after the relevant position to reflect the change from original amino acid (before the position) to changed amino acid (after position). For example, A19T means that amino acid alanine at position 19 is changed to threonine.

The terms “polypeptide variant” and “polypeptide mutant” as used herein refers to a polypeptide that comprises an amino acid sequence wherein one or more amino acid residues are inserted into, deleted from and/or substituted into the amino acid sequence relative to another polypeptide sequence. In certain embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Variants of the present disclosure include fusion proteins.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In certain embodiments, the % identity is selected from, e.g., at least 60%, at least 65%, at least 70%, at least 75%, at least 80%, at least 85%, at least 90%, at least 95%, or at least 99% or more sequence identity to a given sequence. In certain embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In certain embodiments, the % homology is selected from, e.g., at least 60%, at least 65%, at least 70%, at least 75%, at least 80%, at least 85%, at least 90%, at least 95%, or at least 99% or more sequence homology to a given sequence. In certain embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. (Id).

In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. A substantially pure polypeptide or protein will typically comprise about 50%, 60%, 70%, 80% or 90% W/W of a protein sample, more usually about 95%, and e.g., will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

A “pharmaceutical composition” refers to a composition suitable for pharmaceutical use in an animal or human. A pharmaceutical composition comprises a pharmacologically and/or therapeutically effective amount of an active agent and a pharmaceutically acceptable carrier. “Pharmaceutically acceptable carrier” refers to compositions that do not produce adverse, allergic, or other untoward reactions when administered to an animal or a human. As used herein “pharmaceutically acceptable carrier” refers to any of the standard pharmaceutical carriers, vehicles, buffers, and carriers, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” refers to the amount of a compound that, when administered to a subject for treating RCC, is sufficient to affect such treatment of the RCC. The “therapeutically effective amount” may vary depending, for example, on the soluble AXL variant polypeptide selected, the stage of the RCC, the age, weight and/or health of the patient and the judgment of the prescribing physician. An appropriate amount in any given instance may be readily ascertained by those skilled in the art or capable of determination by routine experimentation.

“Inhibitors,” “activators,” and “modulators” of AXL or its ligand GAS6 are used to refer to inhibitory, activating, or modulating molecules, respectively, identified using in vitro and in vivo assays for receptor or ligand binding or signaling, e.g., ligands, receptors, agonists, antagonists, and their homologs and mimetics. The compounds having the desired pharmacological activity may be administered in a physiologically acceptable carrier to a host to modulate AXL/GAS6 function. The therapeutic agents may be administered in a variety of ways, orally, topically, parenterally e.g. intravenous, subcutaneously, intraperitoneally, by viral infection, intravascularly, etc. Intravenous delivery is of particular interest. Depending upon the manner of introduction, the compounds may be formulated in a variety of ways. The concentration of therapeutically active compound in the formulation may vary from about 0.1-100 wt. %.

“Dosage unit” refers to physically discrete units suited as unitary dosages for the individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having RCC. Subjects may be human, but also include other mammals, particularly those mammals useful as laboratory models for human disease, e.g. mouse, rat, etc.

As used herein, the terms “treatment,” “treating,” and the like, refer to administering an agent, or carrying out a procedure for the purposes of obtaining an effect. The effect may be prophylactic in terms of completely or partially preventing a disease or symptom thereof and/or may be therapeutic in terms of effecting a partial or complete cure for a disease, and/or symptoms of the disease. “Treatment,” as used herein, covers any treatment of any virus infection or exposure in a mammal, particularly in a human, and includes: (a) preventing the infection; (b) inhibiting the infection, i.e., arresting its development; and (c) relieving the disease, i.e., causing regression of infection.

Treating may refer to any indicia of success in the treatment or amelioration or prevention of RCC, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions. The term “therapeutic effect” refers to the reduction, elimination, or prevention of the disease, symptoms of the disease, or side effects of the disease in the subject.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010)).

As used herein, the term “progression free survival” (PFS) means the time period for which a subject having a disease survives, without a significant worsening of the disease state. Progression free survival may be assessed as a period of time in which there is no progression of tumor growth and/or wherein the disease status of a patient is not determined to be a progressive disease.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“In combination with”, “combination therapy” and “combination products” refer, in certain embodiments, to the concurrent administration to a patient of a second therapeutic and the soluble AXL polypeptides as used herein. In some embodiments, the combination products are administered non-concurrently. When administered in combination, each component can be administered at the same time or sequentially in any order at different points in time. Thus, each component can be administered separately but sufficiently closely in time so as to provide the desired therapeutic effect. In some embodiments, the soluble AXL polypeptide may offer additive or synergistic benefit to the therapeutic activity of the second therapeutic.

“Concomitant administration” of a known RCC therapeutic drug with a pharmaceutical composition of the present invention means administration of the drug and AXL variant at such time that both the known drug and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

### EXEMPLARY EMBODIMENTS

AXL, MER, Tyro3 and GAS6, as well as related pathways, have been described in WO2011/091305, as well as U.S. application Ser. Nos. 13/554,954 and 13/595,936; all of which are incorporated herein by reference in their entireties for all purposes. The AXL receptor and its activating ligand, GAS6, are important drivers of metastasis and therapeutic resistance in human cancers. This signaling axis represents an attractive target for therapeutic intervention, but the strong picomolar binding affinity (14-33 pM) between endogenous GAS6 and AXL and the promiscuity of small molecule AXL inhibitors has historically presented a barrier to specific and potent inhibition of AXL. AVB-S6-500 is a highly sensitive and specific inhibitor of AXL, with apparent affinity of 93-324 femtomolar to GAS6, which is approximately 200-fold higher affinity than wild-type (WT) AXL. AVB-S6-500 binds GAS6, the sole ligand of AXL, inhibiting its interaction with AXL, thereby dramatically reducing AXL signaled invasion and migration of highly metastatic cells in vitro and inhibiting metastatic disease in preclinical models of aggressive human cancers.

The intrinsic and acquired resistance of ccRCC has been associated with the AXL receptor. 75-85% of all RCCs are classified as ccRCC, which results from mutations in von Hippel-Lindau (VHL), a critical component of the cellular oxygen-sensing pathway. The AXL receptor has been associated with ccRCC. For example, AXL overexpression has been associated with the development of resistance to VEGF receptor inhibitors in preclinical models of ccRCC. AXL overexpression is strongly correlated with ccRCC patient prognosis and survival (Rankin, E K, PNAS, 13373-13378, 2014). Additional recently published data demonstrated that AXL plays a role in ccRCC tumor vascular density and growth at the kidney, suggesting a role for AXL in mediating the angiogenic potential of ccRCC cells (Xiao et al., Cancer Research, 2019).

The present inventors evaluated AVB-S6-500 in a SN12L1 metastatic ccRCC model. Following injection of tumor cells, mice were divided into two treatments groups: control and 5 mg/kg intraperitoneal (i.p.) AVB-S6-500. Treatments began 7 days post inoculation and occurred twice weekly for 8 weeks. A significant reduction in lung tumor burden of mice treated with 5 mg/kg AVB-S6-500 was observed relative to those in the control group. Results also showed no overt toxicity as assessed by bodyweight and clinical observation.

AVB-S6-500 was also evaluated in a patient derived xenograft (PDX) model for ccRCC, where mice were treated with vehicle, AVB-S6-500 and pazopanib, or a combination of AVB-S6-500 and pazopanib. AVB-S6-500 significantly reduced the specific growth rate and weight of tumors in animals in combination with pazopanib. These data suggest that AVB-S6-500 may also be helpful in restoring sensitivity to TKI therapies, validating the rationale for combination therapy with AXL-GAS6 inhibitors and more specifically, the pursuit of clinical evaluation of AVB-S6-500 as a combination treatment with the current standards of care for ccRCC.

The present inventors have previously evaluated PK and toxicokinetics (TK) of AVB-S6-500 and concentrations of GAS6 (PD) in mice (intraperitoneal and i.v. routes) and monkeys (i.v. route) following single and repeat dosing. The PK profile of AVB-S6-500 is compatible with target-mediated drug disposition (TMDD) with 2 parallel elimination paths: normal clearance of IgG and 2nd order that is saturable and fits the typical two compartment model. At low doses in the cynomolgus monkey (below 5 mg/kg), clearance is high and half-life is short, but at doses above 5 mg/kg, clearance is lower, half-life is longer, and volume of distribution is larger. Using the TMDD model (Dirks, N., Clinical Pharmacokinetics, 633-659, 2010), the human dose estimated to be efficacious may range from 1 mg/kg (to ensure GAS6 levels remain at least 90% less than baseline) to 20 mg/kg (to ensure 99% abrogation of GAS6 and allowing for a three-fold increase in GAS6 levels in cancer patients relative to normal levels).

In a 3-month GLP cynomolgus monkey study, once weekly administration of AVB-S6-500, as a 30-minute i.v. infusion at doses of 50, 100 and 150 mg/kg/dosed weekly was well tolerated and resulted in a NOAEL of AVB-S6-500 in cynomolgus monkeys of at least 150 mg/kg/dose. There were no mortalities, toxicologically significant treatment-related clinical signs or effects on body weights, clinical observations, urinalysis parameters, organ weights, ophthalmology and no macroscopic or microscopic observations of significance related to the administration of AVB-S6-500 at doses up to 150 mg/kg/dose. The non-dose dependent clinical pathology changes seen are consistent with an immune response in monkeys to the human AVB-S6-500 protein.

The present inventors previously evaluated AVB-S6-500 in a single-blind, placebo-controlled, first-in-human, Phase 1 Single Ascending Dose (SAD) and Repeat Dose (RD) study in healthy volunteers. Single dose cohorts of 1, 2.5, 5, and 10 mg/kg were evaluated as well as one RD cohort dosed with 5 mg/kg once weekly for four weeks. Subjects were treated with either Placebo (normal saline) or AVB-S6-500 given as i.v. infusions over 60 minutes. AVB-S6-500 was well tolerated at all doses. There were no dose-related adverse events, no serious adverse events, and, as expected, a maximum tolerated dose was not reached. Any adverse events based on laboratory values being outside of normal range were transient and not dose related.

The present inventors are currently evaluating AVB-S6-500 in the Phase 1b portion of a P1b/2 randomized, controlled study in combination with either pegylated liposomal doxorubicin (PLD) or paclitaxel in patients with platinum-resistant, recurrent ovarian cancer. 40 patients have been dosed with AVB-S6-500 at 10 mg/kg (q2w) and there have been no dose limiting toxicities. Higher dose levels of AVB-S6-500 (15 mg/kg and 20 mg/kg) are being enrolled (in combination with paclitaxel and PLD) in Phase 1b expansion cohorts to determine the optimal recommended Phase 2 dose. The PK of AVB-S6-500 are consistent with target-mediated drug disposition (TMDD). In platinum-resistant ovarian cancer patients, preliminary data from Phase 1b indicate a potential exposure-response relationship. If this relationship reflects the extent of engagement of the sink by AVB-S6-500, simulations suggest that a dose of 20 mg/kg (the highest dose to be evaluated in that study) should provide optimal engagement of the sink and therefore, represent the dose level most likely to provide benefit.

Cabozantinib is a kinase inhibitor indicated for the treatment of patients with advanced ccRCC who have received prior antiangiogenic therapy. In vitro biochemical and/or cellular assays have shown that cabozantinib inhibits the tyrosine kinase activity of MET, VEGFR-1, -2 and -3, AXL, RET, ROS1, TYRO3, MER, KIT, TRKB, FLT-3, and TIE-2. Given the preclinical data. CABOMETYX™ (Cabozantinib S-Malate oral tablets) has been approved by the Food and Drug Administration (FDA) in the United States for the treatment of advanced renal cell carcinoma (RCC) who have received prior antiangiogenic therapy on Apr. 25, 2016. Cabozantinib is formulated as the L-malate salt of N-(4-{[6,7-bis(methyloxy)quinolin-4-yl]oxy}phenyl)-N′-(4-fluorophenyl)cyc-lopropane-1,1-dicarboxamide (see, e.g. WO 2005/030140, the entire contents of which is incorporated herein by reference).

Given the preclinical data on AVB-S6-500 impact on AXL-directed MET and VEGF pharmacology and the ability of AXL inhibition to restore VEGFi sensitivity in two models, the present inventors hypothesize that AVB-S6-500 may offer additive or synergistic benefit to the therapeutic activity of cabozantinib. For example, plasma concentrations of GAS6 have been shown to significantly increase upon treatment with cabozantinib (Leibowitz-Amit et el., J Trans Med., 14:12, 2016) and therefore there may be benefit in lowering GAS6 in combination with cabozantinib treatment. Additionally, as a protein directed specifically to bind GAS6, AVB-S6-500 is not expected to have the adverse effects and off-target activities associated with small molecule kinase inhibitors and therefore is not predicted to have drug-drug interactions with cabozantinib.

Methods of the present invention include treating or preventing RCC, by administering a soluble AXL variant polypeptide as described herein. In one aspect, the present invention provides methods for the treatment of RCC, comprising the administration of a soluble AXL polypeptide, according to a regimen determined to achieve stable disease/response and longer progression free survival (PFS) as compared to control.

In another aspect, the present invention provides methods for the treatment of advanced clear cell renal cell carcinoma (ccRCC) in a human patient, comprising the administration of a soluble AXL polypeptide in combination with cabozantinib according to a regimen determined to achieve stable disease/response and longer progression free survival as compared to control. In some embodiments, the soluble AXL polypeptide may offer additive or synergistic benefit to the therapeutic activity of cabozantinib.

In another aspect, the present invention provides methods for the treatment of advanced clear cell renal cell carcinoma (ccRCC) in a human patient who has received prior anti-angiogenic therapy, comprising the administration of a soluble AXL polypeptide in combination with cabozantinib, wherein progression-free survival (PFS) and one or both of overall survival (OS) and objective response rate (ORR) are extended as compared to patients who have received prior anti-angiogenic therapy. In some embodiments, the prior anti-angiogenic therapy is selected from the group consisting of axitinib, pazopanib, sorafenib, sunitinib, everolimus, temsirolimus, bevacizumab, interleukins, interferon-α, peginterferon, nivolumab, and atezolizumab. In some embodiments, the soluble AXL polypeptide may offer additive or synergistic benefit to the therapeutic activity of cabozantinib.

In some embodiments, the methods prolong progression free survival as compared to control. In some embodiments, the methods prolong overall survival as compared to control. In some embodiments, the methods achieve improved progression free survival as compared to control. In some embodiments, the methods achieve improved time to second subsequent therapy as compared to control. In some embodiments, the methods have been determined to not have a detrimental effect on Quality of Life as determined by FOSI and/or EQ-5D-5L.

In some embodiments, the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0 mg/kg, of about 19.5, of about 20.0 mg/kg, of about 25.0 mg/kg, of about 30.0 mg/kg, of about 35.0 mg/kg, and of about 40.0 mg/kg. In some embodiments, the soluble AXL variant polypeptide will be given as IV infusion over 30 or 60 or 90 minutes at a weekly dose of 20 mg/kg. In some embodiments, the soluble AXL variant polypeptide will be given as IV infusion over 30 or 60 or 90 minutes at a weekly dose of 10 mg/kg. In some embodiments, the soluble AXL variant polypeptide will be given as IV infusion over 30 or 60 or 90 minutes at a weekly dose of 5 mg/kg. In some embodiments, the soluble AXL variant polypeptide will be given as IV infusion over 30 or 60 or 90 minutes at a weekly dose of 2.5 mg/kg. In some embodiments, the soluble AXL variant polypeptide will be given as IV infusion over 30 or 60 or 90 minutes at a weekly dose of 1 mg/kg. In some embodiments, the soluble AXL variant polypeptide will be given as IV infusion over 30 or 90 minutes at a dose of 25 mg/kg every 14 days. In some embodiments, the soluble AXL variant polypeptide will be given as IV infusion over 30 or 90 minutes at a dose of 20 mg/kg every 14 days. In some embodiments, the soluble AXL variant polypeptide will be given as IV infusion over 30 or 90 minutes at a dose of 15 mg/kg every 14 days. In some embodiments, the soluble AXL variant polypeptide will be given as IV infusion over 30 or 60 or 90 minutes at a dose of 10 mg/kg every 14 days. In some embodiments, the soluble AXL variant polypeptide will be given as IV infusion over 30 or 60 or 90 minutes at a dose of 5 mg/kg every 14 days. In some embodiments, the soluble AXL variant polypeptide will be given as IV infusion over 30 or 60 or 90 minutes at a dose of 2.5 mg/kg every 14 days. In some embodiments, the soluble AXL variant polypeptide will be given as IV infusion over 30 or 60 minutes at a dose of 1 mg/kg every 14 days.

In some embodiments, the dose of cabozantinib to be co-administered to the patient along with the soluble AXL variant polypeptide is selected from the group consisting of 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, and 5 mg, once daily with fasting.

In still some embodiments, therapeutic entities of the present invention are often administered as pharmaceutical compositions comprising an active therapeutic agent, i.e., and a variety of other pharmaceutically acceptable components. (See Remington's Pharmaceutical Science, 15.sup.th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. In some embodiments, the composition comprises polypeptide at 1 mg/mL, formulated in aqueous buffer consisting of 10 mM Tris, 210 mM sucrose, 51 mM L-arginine, 0.01% polysorbate 20, adjusted to pH 7.4 with HCl or NaOH.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

The pharmaceutical compositions are generally formulated as sterile, substantially isotonic and in full compliance with all Good Manufacturing Practice (GMP) regulations of the U.S. Food and Drug Administration. Preferably, a therapeutically effective dose of the polypeptide compositions described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

### Example 1

### A Phase 1b/2 Randomized, Study of AVB-S6-500 in Combination with Cabozantinib Versus Cabozantinib Alone in Patients with Advanced Cell Renal Cell Carcinoma that have Received Front-Line Treatment

AVB-S6-500 (supplied as 20 mg/mL AVB-S6-500, 0.01% polysorbate, 10 mM phosphate, 9% sucrose, ph 7.0) will be given by i.v. infusion (150 mL total volume) over 1 hour (h), on Day 1 and 15 of each 28-day treatment cycle. AVB-S6-500 dosing may continue if cabozantinib is discontinued. Cabozantinib will be used as per standard of care (SOC), i.e., at 60 mg orally once daily at a distance from meals (do not eat at least 2 h before or 1 h after taking cabozantinib), with ability to decrease dose per labeling if patient does not tolerate 60 mg.

### Phase 1b

The Phase 1b portion of this protocol is a multicenter, open-label, 3+3 dose escalation study to evaluate safety, tolerability, PK, and PD of AVB-S6-500 combined with cabozantinib in subjects with advanced ccRCC who have received front-line treatment. The primary objective of the Phase 1b is to evaluate the safety and tolerability of AVB-S6-500 in combination with cabozantinib in subjects with advanced ccRCC. The secondary objectives of the Phase 1b study are: 1) to identify the recommended Phase 2 dose (RP2D) of AVB-S6-500 in combination with cabozantinib; 2) to evaluate the efficacy of AVB-S6-500 in combination with cabozantinib; 3) to evaluate the pharmacokinetic (PK) profile of AVB-S6-500 administered in combination with cabozantinib; 4) to evaluate pharmacodynamic GAS6 serum levels before and during treatment; and 5) to evaluate potential immunogenicity of AVB-S6-500.

3 to 6 subjects (per dose level) will be administered AVB-S6-500 in combination with cabozantinib. A data monitoring committee (DMC) will meet to review the data after 3 subjects (or after 6 subjects, if applicable) have completed 28 days of treatment. In the initial cohort, AVB-S6-500 will be administered every 2 weeks at an initial dose level of 15 mg/kg in combination with cabozantinib given at a dose of 60 mg daily. Subsequent cohorts will receive 20 mg/kg AVB-S6-500 and then 25 mg/kg AVB-S6-500, respectively, in combination with cabozantinib given at a dose of 60 mg daily. While the recommended dose of cabozantinib is 60 mg daily in each dose level used in this study, cabozantinib may be given at lower doses or dose reduced when needed (i.e., for hepatic impairment, in the setting of anticipated drug-drug interactions with concomitant medications, or to manage adverse events associated with cabozantinib) as per the package insert and/or institutional practice.

Tolerability will be evaluated by the DMC after review of open-label safety data in each cohort (in a 3+3 design format) from the first 28 days. The determination of tolerability of the AVB-S6-500 dose in combination with cabozantinib will be made by comparison of the frequency and duration of all AEs, Grade ≥3 AEs and the SAEs in the combination regimen to the known toxicity profile of cabozantinib. This enrollment and safety review process will repeat until evaluation of the 20 mg/kg q2W dose level has completed.

### Phase 2

The Phase 2 portion of this protocol is a multicenter, randomized, 2-arm, open-label study to compare the efficacy and tolerability of AVB-S6-500 in combination with cabozantinib versus cabozantinib alone in subjects with advanced ccRCC that have received front-line treatment. A total of approximately 45 subjects will be enrolled and randomized 2:1 into one of the two treatment arms: 1) AVB-S6-500+cabozantinib (N=30) and 2) cabozantinib alone (N=15). The Phase 2 dose will be a dose/dosing regimen of AVB-S6-500 that is deemed safe/tolerable in combination with cabozantinib and demonstrates desirable PK/PD profile on evaluation of 28-day data from the Phase 1b study for use in future studies.

The primary objective of the Phase 2 study is to compare progression-free survival (PFS) in advanced ccRCC subjects treated with AVB-S6-500 in combination with cabozantinib versus cabozantinib alone. The secondary objectives of the Phase 2 study are: 1) to evaluate additional efficacy endpoints (e.g., ORR, DOR, DCR, OS); 2) to evaluate the safety and tolerability of AVB-S6-500 in combination with cabozantinib; 3) to evaluate the PK and PD profile of AVB-S6-500; and 4) to evaluate the immunogenicity of AVB-S6-500. PFS is defined as the interval between randomization and the first radiologically documented disease progression or death, whichever comes first. The objective response rate (ORR) is defined as the proportion of subjects who have a partial or complete response to therapy. Evaluation of the duration of response (DOR) will include subjects with a complete or partial response (defined by RECIST criteria) and is measured from the date of response until the cancer progresses. The disease control rate (DCR) is defined as the proportion of subjects who have a complete or partial response or maintain stable disease.

Impact of treatment on PFS will be evaluated using Product-Limit estimates of the PFS event distributions for each treatment group using the FAS. In addition, medians and their corresponding 95% CIs (Brookmeyer and Crowley 1982) will be provided by treatment group. The PFS will also be modeled in a stratified Cox proportional hazards model, with stratification by IMDC risk group, and with parametric factors for treatment group, prior VEGFi treatment (yes, no), GAS6, and AXL to provide hazard ratios for each factor, and their corresponding 95% CIs. The DOR and OS secondary endpoints will be analyzed analogous to PFS.

Antineoplastic activity will also be assessed for each of the categorical secondary endpoints of confirmed ORR and DCR. Confirmed ORR will be summarized as the proportion of treated subjects who achieve a best response of CR or PR, including the corresponding two-sided 95% exact CIs (Clopper-Pearson method) (Clopper and Pearson 1934). The DCR will be analyzed analogous to confirmed ORR.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize or be able to ascertain using no more than routine experimentation, many equivalents to the specific embodiments of the invention described herein. Such equivalents are intended to be encompassed by the appended claims.

### SEQUENCE LISTINGS

The nucleic and amino acid sequences listed in the accompanying sequence listing are shown using standard letter abbreviations for nucleotide bases and three letter code for amino acids, as defined in 37 C.F.R. 1.822.

Human AXL polypeptide amino acid sequence
SEQ ID NO: 1
MGRVPLAWCLALCGWACMAPRGTQAEESPFVGNPG
NITGARGLTGTLRCQLQVQGEPPEVHWLRDGQILE
LADSTQTQVPLGEDEQDDWIVVSQLRITSLQLSDT
GQYQCLVFLGHQTFVSQPGYVGLEGLPYFLEEPED
RTVAANTPFNLSCQAQGPPEPVDLLWLQDAVPLAT
APGHGPQRSLHVPGLNKTSSFSCEAHNAKGVTTSR
TATITVLPQQPRNLHLVSRQPTELEVAWTPGLSGI
YPLTHCTLQAVLSNDGMGIQAGEPDPPEEPLTSQA
SVPPHQLRLGSLHPHTPYHIRVACTSSQGPSSWTH
WLPVETPEGVPLGPPENISATRNGSQAFVHWQEPR
APLQGTLLGYRLAYQGQDTPEVLMDIGLRQEVTLE
LQGDGSVSNLTVCVAAYTAAGDGPWSLPVPLEAWR
PGQAQPVHQLVKEPSTPAFSWPWWYVLLGAVVAAA
CVLILALFLVHRRKKETRYGEVFEPTVERGELVVR
YRVRKSYSRRTTEATLNSLGISEELKEKLRDVMVD
RHKVALGKTLGEGEFGAVMEGQLNQDDSILKVAVK
TMKIAICTRSELEDFLSEAVCMKEFDHPNVMRLIG
VCFQGSERESFPAPVVILPFMKHGDLHSFLLYSRL
GDQPVYLPTQMLVKFMADIASGMEYLSTKRFIHRD
LAARNCMLNENMSVCVADFGLSKKIYNGDYYRQGR
IAKMPVKWIAIESLADRVYTSKSDVWSFGVTMWEI
ATRGQTPYPGVENSEIYDYLRQGNRLKQPADCLDG
LYALMSRCWELNPQDRPSFTELREDLENTLKALPP
AQEPDEILYVNMDEGGGYPEPPGAAGGADPPTQPD
PKDSCSCLTAAEVHPAGRYVLCPSTTPSPAQPADR
GSPAAPGQEDGA
Exemplary soluble AXL polypeptide-Fc fusion.
SEQ ID NO: 2
EESPFVSNPGNITGARGLTGTLRCQLQVQGEPPEV
HWLRDGQILELVDSTQTQVPLGEDEQGDWIVASQL
RITSLQLSDTGQYQCLVFLGHQTFVSQPGYVRLEG
LPYFLEEPEDRTVAANTPFNLSCQAQGPPEPVDLL
WLQDAVPLATAPGHGPQRSLHVPGLNKTSSFSCEA
HNAKGVTTSRTATITVLPQQGGGGSDKTHTCPPCP
APELLGGPSVFLFPPKPKDTLMISRTPEVTCVVVD
VSHEDPEVKFNWYVDGVEVHNAKTKPREEQYNSTY
RVVSVLTVLHQDWLNGKEYKCKVSNKALPAPIEKT
ISKAKGQPREPQVYTLPPSREEMTKNQVSLTCLVK
GFYPSDIAVEWESNGQPENNYKTTPPVLDSDGSFF
LYSKLTVDKSRWQQGNVFSCSVMHEALHNHYTQKS
LSLSPG

1-11. (canceled)

12. A method for treating advanced clear cell renal cell carcinoma (ccRCC) in a patient, comprising the administration of a soluble AXL polypeptide in combination with cabozantinib according to a regimen determined to achieve stable response and longer progression free survival (PFS) as compared to a patient administered cabozantinib alone.

13. The method according to claim 12, wherein the effects of the soluble AXL polypeptide and cabozantinib are synergistic.

14. The method according to claim 12, wherein the patient had received prior anti-angiogenic therapy.

15. The method according to claim 14, wherein the prior anti-angiogenic therapy is selected from the group consisting of axitinib, pazopanib, sorafenib, sunitinib, everolimus, temsirolimus, bevacizumab, interleukins, interferon-α, peginterferon, nivolumab, and atezolizumab.

16. The method according to claim 12, wherein the soluble AXL variant polypeptide lacks the AXL transmembrane domain; lacks a functional fibronectin (FN) domain; has one or more than one Ig1 domain and, optionally, one or more than one Ig2 domain; and has a set of amino acid modifications of the wild-type AXL sequence (SEQ ID NO:1), selected from the group consisting of:
1) Gly32Ser, Asp87Gly, Val92Ala, and Gly127Arg,
2) Glu26Gly, Val79Met, Val92Ala, and Gly127Glu; and
3) Gly32Ser, Ala72Val, Asp87Gly, Val92Ala, and Gly127Arg;
wherein said modification increases the affinity of the AXL polypeptide binding to Growth arrest-specific protein 6 (GAS6).

17. The method according to claim 12, wherein the soluble AXL variant polypeptide is fused to an Fc region.

18. The method according to claim 1, wherein the dose of the soluble AXL variant polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 5.5, of about 6.0, of about 6.5, of about 7.0, of about 7.5, of about 8.0, of about 8.5, of about 9.0, of about 9.5, of about 10.0 mg/kg, of about 10.5, of about 11.0, of about 11.5, of about 12.0, of about 12.5, of about 13.0, of about 13.5, of about 14.0, of about 14.5, of about 15.0, of about 15.5, of about 16.0, of about 16.5, of about 17.0, of about 17.5, of about 18.0, of about 18.5, of about 19.0 mg/kg, of about 19.5, of about 20.0 mg/kg, of about 25.0 mg, and of about 30.0 mg/kg.

19. The method according to claim 1, wherein the dose of cabozantinib is selected from the group consisting of 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, and 5 mg, once daily with fasting.

20. The method according to claim 19, wherein the dose of the soluble AXL variant polypeptide is 15 mg/kg given bi-weekly, and the dose of cabozantinib is 60 mg once daily with fasting.

21. The method according to claim 19, wherein the dose of the soluble AXL variant polypeptide is 20 mg/kg given bi-weekly, and the dose of cabozantinib is 60 mg once daily with fasting.

22. The method according to claim 19, wherein the dose of the soluble AXL variant polypeptide is 25 mg/kg given bi-weekly, and the dose of cabozantinib is 60 mg once daily with fasting.
